(19) United States
(12) Patent Application Publication
(10) Pub. No.: US 2012/0003187 A1
(43) Pub. Date: Jan. 5, 2012

(54) BIOLOGICAL MEDIUM FOR PRESERVING A PREPARATION OF INSULIN-SECRETING CELLS

(75) Inventors: Julie Kerr-Conte, Lille (FR); Bruno Delorme, Marcq-En-Baroeul (FR)

(73) Assignees: MACO PHARMA, Mouvaux (FR); UNIVERSITE DU DROIT ET DE LA SANTE DE LILLE 2, Lille (FR); CENTRE HOSPITALIER REGIONAL UNIVERSITAIRE DE LILLE, Lille Cedex (FR)

(21) Appl. No.: 13/101,237

(22) Filed: May 5, 2011

(30) Foreign Application Priority Data
May 5, 2010 (FR) 10 01936

(51) Int. Cl. A61K 35/39; C12N 9/50; A61P 1/18; C12N 5/071

(52) U.S. Cl. 424/93.7; 435/374; 435/219

(57) ABSTRACT

A method for preserving a preparation of insulin-secreting cells includes the step of placing the preparation of insulin-secreting cells in contact with a biological medium, the biological medium including a nutritive product including albumin, and further includes a peptone or a mixture of peptones in sufficient quantity to preserve the insulin-secreting cells.

### BACKGROUND

The invention relates to a method for preserving a preparation of insulin-secreting cells intended to be transplanted into a patient.

The invention applies, in particular, to the field of cell therapy for pancreatic diseases, such as diabetes, which aims to prepare pancreatic islets or Langerhan's islets from a pancreas taken from a donor who is brain dead or whose heart has stopped, or a living donor (i.e., partial or complete pancreatectomy). In the latter case, allogenic or autologous therapies are foreseeable. These islets, which include insulin-secreting beta cells, are then transplanted from a receiving patient for the purpose of restoring the endogenous secretion of insulin in the patient.

Therefore, these cell therapies currently represent an attractive alternative for treating the most severe types of diabetes. An increasing number of teams propose a total pancreatectomy in patients suffering from IPMT (Intraductal Papillary Mucinous Tumour) of the pancreas. However, the absence of endogenous secretion of insulin and glucagon seriously disrupts the metabolism of these patients, with a short and long-term vital risk. Restoring insulin secretion by autologous transplantation of pancreatic islets is therefore a priority in this disease.

The pancreatic islets are virtually isolated via enzyme and mechanical digestion from a pancreas, and then purified by density gradient. The islets are next transplanted directly into the patient or else preserved for 1 to 3 days prior to the transplantation thereof.

This pre-transplantation preservation step has proven beneficial from the metabolic and immunological standpoint. As a matter of fact, the time spent preserving the islets can enable the receiver to be prepared for the graft. Furthermore, this preservation step likewise enables the required quality controls to be carried out on the islets prior to the perfusion thereof. Finally, packaging the islets in a preservation medium enables same to be transported to one centre or another. Not surprisingly, culturing/preservation of the islets has recently been adopted by the majority of pancreatic islet transplantation centres for the purpose of enhancing the safety and practical aspect of transplanting the islets.

However, with the current static preservation methods, between 40 and 60% of the islets are lost during the first 24-hour culture period after isolation.

The document EP-A1-2,119,352 proposes a method for preserving pancreatic islets by continuously refreshing the culture medium. The culture medium used in this method, which provides the best results, is a medium including the modified CMRL base medium (Cambrex) supplemented by AB serum and Stem Ease® (Abcys).

However, the use of the serum in the culture medium produces a risk in terms of safety since the composition thereof is undefined and variable from one batch to another and, being of animal origin, there is risk of contamination, in particular viral and proteinaceous, e.g., by the prion.

Serum-free culture media for culturing and/or preserving pancreatic islets have been developed.

For example, the document WO-03/044181 describes a culture medium for improving the functionality and viability of pancreatic islets, which includes vitamin E and nicotinamide, and in which the serum is replaced by albumin. The medium further includes insulin, transferrin, selenium, linoleic acid, sodium pyruvate, zinc sulphate or chloride, a HEPES buffer and L-glutamine.

Other serum-free media for pancreatic islets have been described by Samuel A. Clark in Endocrinology 126(4), 1895-1903 (1990). In this document, various serum-free culture media are tested, namely serum-free base media supplemented with a proteose-peptone as a serum substitute, tranferrin or one or more hormones.

In this same article, the proteose-peptone was next substituted with the albumin, ethanolamine and phosphoethanolamine combination to obtain a specific, peptone-free and serum-free medium including an RPMI-1640 base medium with 0.1% human seric albumin, transferrin, the thyroid hormone (T3), PRL, IGF-1, ethanolamine, phosphoethanolamine and L-glutamine.

This article does not describe any medium including albumin and a peptone. Furthermore, the peptone is used in the same capacity as albumin, for the nutritive properties thereof, as a serum substitute.

Other media for pancreatic islets are described in literature. For example, the document WO2005/120576 proposes adding 50 M of alpha-tocopherol to a culture medium in order to reduce the damage caused to the pancreatic islets by anoxia. The document US2007/0196810 proposes adding a polymerised haemoglobin to the culture medium used to isolate the islets of the pancreas. And, in the document US2006/0246582, a peptide analogous to the laminine A chain is added to the culture medium for the islets in order to enable the culture density of the islets to be increased up to 300 islets/mL.

These media, however, remain insufficient to obtain an acceptable cultured islet survival rate.

The document WO 2008/009642 describes a serum-free culture medium intended for culturing cells used, in particular, for producing proteins of interest. The medium includes a base medium and a recombinant albumin as a serum substitute. The medium can further include insulin, sodium selenite, glutamine, transferrin, peptone, ethanolamine, fetuin, vitamins, lipoproteins, fatty acids and/or amino acids in order to improve the growth and productivity of the cells such as the hybridomas. More specifically, the peptone is combined with the recombinant albumin, the insulin and optionally the transferrin. In this medium, the peptone is still used as a nutritive element to support the growth and expansion of the cells.

### SUMMARY OF THE INVENTION

The invention proposes a method for preserving insulin-secreting cells by means of a serum-free biological medium approved for clinical use. The serum-free medium does not contain any element of non-human animal origin and achieves the same performance levels in terms of cell viability and functionality as a medium supplemented with serum.

To that end and according to a first aspect, the invention proposes a method for preserving a preparation of insulin-secreting cells including the step of:

placing said preparation of insulin-secreting cells in contact with a biological medium, said biological medium including a nutritive product including albumin, and further including a peptone or a mixture of peptones in sufficient quantity to preserve the insulin-secreting cells.

According to a second aspect, the invention relates to a composition including (a) a preparation of insulin-secreting cells and (b) a biological medium with albumin, and a peptone or mixture of peptones.

Another aspect relates to a drug for treating pancreatic diseases including a cell composition according to the second aspect.

### BRIEF DESCRIPTION OF THE DRAWINGS

The invention will be understood from the following description.

FIG. 1 shows the number of J5 islet equivalents (IE) in three different media.

FIG. 2 shows the quantity of insulin (in μUI/40 IE/H) secreted by the islets in three different media in the presence of 20 mM of glucose.

FIG. 3 shows the quantity of insulin (in μUI/40 IE/H) secreted by the islets in three different media in the presence of 2.8 mM of glucose.

FIG. 4 shows the stimulation index (SI) of the islets in three different media.

FIG. 5 shows the quantity of intracellular insulin per islet (in μUI/40/IE/H) in three different media.

FIG. 6 shows the assaying of the apoptosis of the islets at 405 nm in three different media.

### DETAILED DESCRIPTION OF THE PREFERRED EMBODIMENT(S)

According to a first aspect, the invention relates to a method for preserving a preparation of insulin-secreting cells. In particular, the method enables short-term preservation of the cells, i.e., of the order of 1 to 7 days, in particular from 1 to 5 days at 37° C. or at 22-25° C.

According to the invention, the preservation method is a method which protects the insulin-secreting cells from the harmful effects of the environment thereof, by maintaining their viability and functionality.

As a second aspect, the invention likewise relates to a composition including an insulin-secreting preparation and a biological medium including albumin and a peptone or a mixture of peptones.

The composition of the invention is capable of keeping the living insulin-secreting cells in good condition and of potentially protecting same from the harmful effects of sampling, isolation and grafting.

The preparation of insulin-secreting cells is in particular a preparation of pancreatic islets or a preparation of other types of cells differentiated as insulin-secreting cells, like adult stem cells (pancreatic, mesenchymatous, hematopoietic, induced pluripotent stem cells (iPS), . . . ).

In particular, the preparation of insulin-secreting cells is a preparation of pancreatic islets derived from a pancreas of a brain dead donor. The islets are isolated by enzyme and mechanical digestion of the pancreas and then purified by density gradient. They are next preserved before being transplanted.

The method and the composition of the invention apply to the islets thus isolated, which are contained in high-purity preparations, in particular of the order of 90%, and medium-purity preparations, but also to preparations of lower purity, such as less than 50%.

Thus, by using such an isolation method, the preparation of secreting cells further includes proteolytic enzymes, which are secreted by the cells and exocrine tissues of the pancreas.

The preservation method and the composition of the invention enable the functionality and viability of the insulin-secreting cells to be maintained. In particular, the cells thus preserved retain the capability thereof to secret insulin. The method and the composition of the invention do not primarily aim to expand or multiply the pancreatic islets but rather to preserve the capability of same to express insulin.

According to the method of the invention, the preparation of insulin-secreting cells is placed in contact with a biological medium which, on the one hand, includes a nutritive product including albumin and, on the other hand, a peptone or a mixture of peptones in sufficient quantity to preserve the insulin-secreting cells.

A composition results from this method, which includes a preparation of insulin-secreting cells and a biological medium which, on the one hand, includes a nutritive product including albumin and, on the other hand, a peptone or a mixture of peptones. This composition enables the insulin-secreting cells to be preserved.

The concentration of the pancreatic islets in the biological medium is between 400 and 40,000 islet equivalents/mL. The islet concentration is advantageously of the order of 500-3,000 islet equivalents/mL.

Due to the fact that the islets may have very different sizes, reference is made to islet equivalents (IE), one islet equivalent corresponding to a standard islet of 150 μm in diameter.

In particular, no biological medium, nutritive product and culture medium contains any serum.

According to one embodiment, the nutritive product includes a serum-free liquid culture medium supplemented with albumin.

The biological medium, for example, includes a CMRL 1066 (In Vitrogen) or MEM (MacoPharma) or Ham's F10, or M199 type base culture medium.

The albumin is advantageously serum albumin, in particular human serum albumin or human recombinant albumin. It replaces the serum used in culture media for research purposes.

Albumin is a protein which promotes the transport of lipid molecules (fatty acids) and/or liposoluble molecules (particularly vitamins), and a some metal ions (Cu2+, Zn2+ and Ca2+).

In a serum-free culture medium, the albumin plays a role in supporting the cell growth of various types of cells, substantially when complexed with a lipid component (fatty acids).

In order to enable the insulin-secreting cells to be preserved, the biological medium includes between 0.1 and 1% by weight/volume of albumin, in particular 0.625% by weight/volume.

Furthermore, the biological medium includes a peptone or a mixture of peptones.

A peptone is a soluble protein substance, which results from the chemical or enzymatic hydrolysis of proteins. The peptones are obtained from milk proteins such as casein, animal proteins derived from muscles or organs, yeasts, or plant proteins.

The peptone used in the method and composition of the invention is advantageously of non-animal origin, so as to avoid the risks associated with animal proteins, like bovine and transmissible spongiform encephalopathies.

For example, the preferred peptone is a plant peptone produced from plants. In particular, the peptone is a pea, wheat, potato, rice, cotton or soy peptone.

With the islet preparation methods, the islet preparations are not pure and still contain up to 50% exocrine cells such as epithelial cells. These exocrine cells contain various proteolytic preproenzymes which are transformed into active proteolytic enzymes, such as trypsine, chymotrypsin and carboxypeptidase, and which are capable of damaging the pancreatic islets and affecting the functionality and viability thereof.

Peptone contains amino acids, peptides, vitamins and essential elements.

In the biological medium, the peptone thus constitutes an alternative target for these proteolytic enzymes, which are then less likely to attack the pancreatic islets.

In the method and composition of the invention, the peptones then constitute a substrate for the proteolytic enzymes of the tissues and/or exocrine cells.

The biological medium includes, in particular, between 0.01 and 10% by weight/volume of peptone, particularly between 0.05 and 5% by weight/volume of peptone and specifically between 0.1% and 1% by weight/volume of peptone. For example, the peptone concentration in the medium is from 0.1% by weight/volume.

This concentration is sufficient to improve the survival and insulin-secreting capability of the insulin-secreting cells.

Furthermore, it has been shown that a significant concentration of albumin, in particular of the order of 5% by weight/volume, enables the apoptosis to be reduced in human islets preserved in-vitro (Artif Cells Blood Substit Immobil Biotechnol. 2008; 36(1): 74-81).

According to the invention, the presence of a peptone in the biological medium enables the apoptosis of the islets to be reduced with a relatively low concentration of albumin, e.g., less than 1% by weight/volume of albumin.

On the other hand, albumin is likewise a target of trypsin and chymotrypsin. It thus strengthens the role assigned to the peptone, of providing a diversion to the proteolytic enzymes of the pancreas.

The peptone used in the method of the invention is advantageously protein-rich. In particular, it includes more than 70% by weight, and preferably more than 80% by weight of proteins.

The protein content of the peptone is obtained by multiplying the proteinaceous nitrogen content of the peptone by 6.25, which is itself calculated by using the Kjeldahl method.

In order to increase the protein load of the biological medium, the peptone has a low degree of hydrolysis.

The pH of the peptone is close to the physiological pH, i.e., around 7.

In particular, the peptone is rich in amino acids having a long aromatic or hydrophobic side chain, such as phenylalanine, tryptophan or tyrosine. As a matter of fact, these amino acids are the preferred target of chymotrypsin, one of the proteolytic enzymes which affect the islets.

Alternatively, the peptone is rich in hydrophobic carboxy-terminal amino acids, such as leucine, phenylalanine and tyrosine. These amino acids are recognized and released by the carboxypeptidase.

The peptone is advantageously rich in leucine, phenylalanine, tyrosine and tryptophan. In particular, the cumulative content of these four amino acids in the peptone is greater than 100 mg/g, and specifically 150 mg/g.

In addition, the peptone is selected to have the lowest possible endotoxin level, in order to prevent any contamination. In particular, the endotoxin level of the peptone is lower than 200 EU/g.

The peptone of plant origin, which meets the relatively high leucine, tyrosine, tryptophan and phenylalanine content criteria, with a protein content greater than 70% by weight, a physiological pH, a low hydrolysis rate and a low endotoxin content, is the pea or potato peptone. The pea peptone is preferred. This pea peptone is obtained by enzymatic hydrolysis of pea meal proteins.

The peptones can be obtained commercially from various manufacturers and/or distributors, such as Solabia, Millipore and Kerry Biosciences, . . . .

The biological medium contains one or more protective elements having an antioxidant activity, in particular so as to prevent damages associated with hypoxia of the beta cells, which results in cell death.

For example, the biological medium contains the N-acetylcysteine-based antioxidant Stem-ease® (Abcys, France).

In another example, the biological medium contains a cocktail of protective elements which includes at least two scavengers of free radicals or antioxidant substances. For example, the free radical scavengers are selected from the group including vitamin derivatives, derivatives having at least one thiol function and linear or cyclic polyols.

Other substances having an antioxidant activity are zinc derivatives, such as zinc sulphate.

According to one particular embodiment, the biological medium further includes polyethylene glycol, and particularly between 0.1 and 3% by weight/volume of polyethylene glycol. Polyethylene glycol has an immunoprotective role.

A quantity less than 1% by weight/volume, and in particular, 0.5% by weight/volume of polyethylene glycol in the biological medium is sufficient to ensure the immunoprotective role thereof.

According to one particular embodiment, the biological medium does not contain any hormones, in particular no transferrin, insulin or growth factors. Since the growth factors used in culture media are generally complex and costly synthetic molecules, a medium devoid of such molecules is therefore a particularly advantageous alternative.

The serum-free biological medium is advantageously a medium for clinical use. The media used for research purposes are media used to cultivate and preserve cells intended exclusively for laboratory studies, i.e., the cells thus cultivated are not introduced into humans. On the contrary, a medium for clinical or therapeutic use, or of a clinical grade, is used to cultivate or preserve cells which will be re-implanted or reintroduced into humans.

In addition, the medium is devoid of any xenogenic product, i.e., belonging to a species other than man. It does not contain any product of animal origin other than man.

The medium is of a clinical grade in that it does not contain any product of animal origin and contains elements approved by the French regulatory authorities.

According to the method of the invention, the cell preparation is placed in contact with the biological medium for a time period of between 1 and 5 days. The biological medium as described above therefore enables short-term preservation of the insulin-secreting cells.

The preservation temperature is between 20° C. and 37° C., and, in particular, it is at room temperature.

The biological medium of the method and composition of the invention is ready for use, and packaged in a container, pouch or flask. It does not require reconstitution in the laboratory or hospital.

The medium contained in the container is sterile. It is advantageously introduced into the container by aseptic filling.

The biological medium containing albumin and at least one peptone can likewise be used during the isolation or extraction of the islets from a pancreas, in particular at the rinsing stage, when the level of exocrine enzymes released from the digested exocrine tissue is particularly high.

It can be used as a transport medium for transporting the islet preparation from one centre to another, or for preserving same for a few days. And it can likewise be used for grafting islets.

The method of the invention for preserving insulin-secreting islets can therefore be implemented during the isolation, extraction, rinsing, culturing, preservation or transport of the cells.

According to another aspect, the invention relates to a drug for treating pancreatic diseases, such as diabetes, said drug including a composition as defined above. The composition includes, in particular, albumin in a nutritive product and a peptone or mixture of peptones.

This composition is used for treating pancreatic diseases such as diabetes. It is then transplanted in humans, e.g., by perfusion.

### Example

Preparations of islets having a purity level of approximately 40% are taken up at the end of the isolation procedure, in three different biological media at a concentration of 15,000 IEQ/175 cm2/30 ml. The islet preparations include approximately 3,000 islet equivalents (IE).

The islets are preserved for 5 days at 37° C., 5% CO2 in three different media, and refreshed at 18-24 h.

| | VC | V0 | V1 |
|---|---|---|---|
| Base medium | CMRL 1066 | CMRL 1066 | CMRL 1066 |
| PEG (%) | | 0.5% | 0.5% |
| AB Serum (%) | 2.5% | | |
| HSA (%) | | 0.625% | 0.625% |
| Pea peptone | | | 0.1% |
| Protective | 0.1 mg/ml | Cocktail | Cocktail |
| elements | Stem- | | |
| | Ease ® | | |

The evaluation is carried out at the end of the fifth day, using the following criteria:

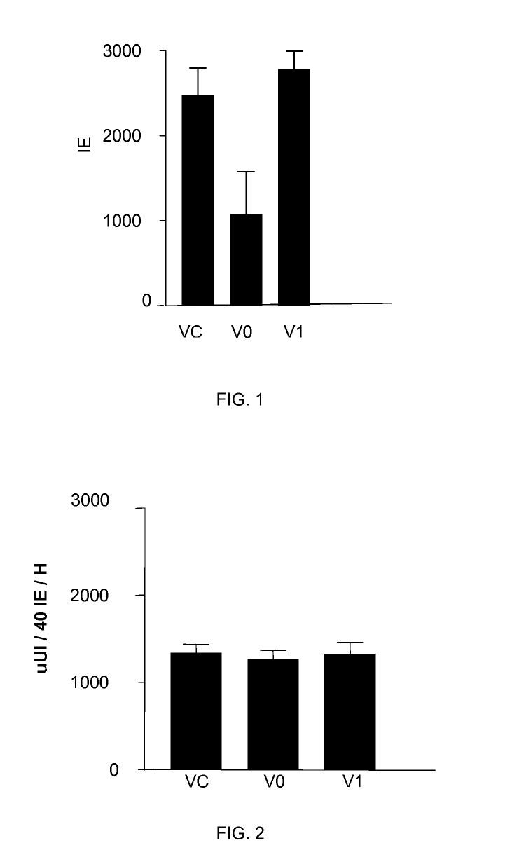

the number of islet equivalents (IE), by counting the islets after dithizone staining (FIG. 1),

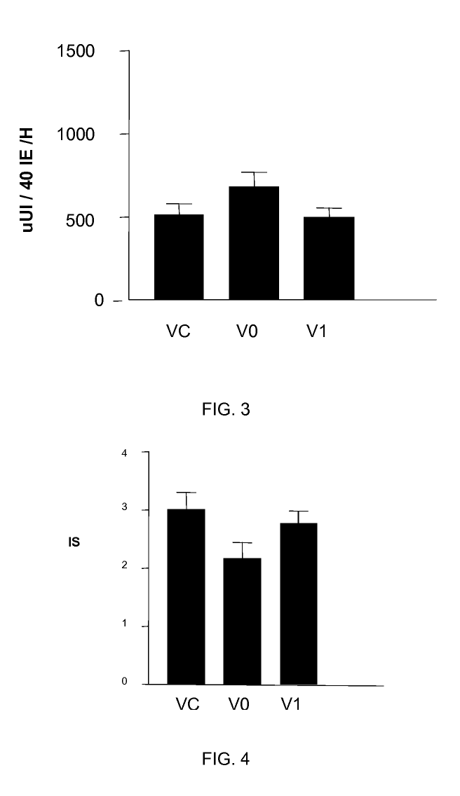

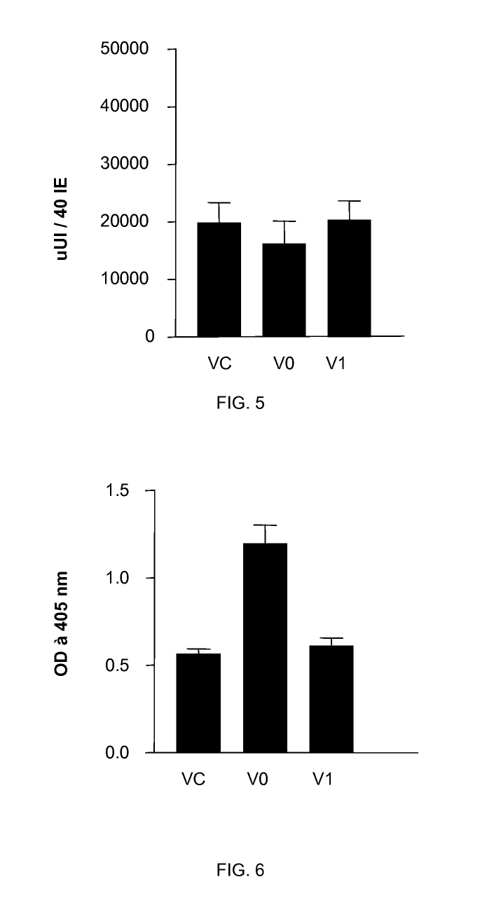

the physiology of the islets, by determining the intracellular quantity (μlU/40 IE) of insulin (FIG. 5), the insulin secretion rate (μlU/40 IE) in response to 20 mM of glucose (FIG. 2) and to 2.8 mM of glucose (FIG. 3), and the stimulation index (FIG. 4),

cell death by assaying (at 405 nm) apoptosis (FIG. 6).

The conventional microscopic assessment of cell viability, purification and yield was carried out by selective staining of the beta cells with dithizone, a zinc chelator, and therefore specific marker for the beta cells, and with Trypan blue, an exclusion stain which marks the dead cells. The dead beta cells appear as violet coloured.

The functional activity of the beta cells was evaluated by the intracellular insulin levels and the secretion of insulin in response to glucose stimulation tests.

Apoptosis is characterised by the fragmentation of the DNA in mono and oligonucleosomes, which can be detected in cell lysates. Quantification of the nucleosomes was carried out using the kit “Cell Death Detection ELISA” by Roche Molecular Biochemicals. The detection principle is based on a sandwich immunoenzymatic assay of the monoclonal antibodies directed against the DNA and histones. The assay was carried out on 3 samples of 160 IE per pancreas.

The insulin content was assayed by a radioimmunological technique using the CT kit by CIS Bio International, on the lysates of 3 samples of 40 IE per pancreas. The insulin secretions were assessed for two consecutive one-hour periods. The first was done in the presence of 2.8 mM of glucose and the second at the stimulating concentration of 20 mM of glucose. The stimulation index is defined as the ratio of the quantities of insulin secreted during these two periods (high over low concentration). The insulin secretions were assessed for 5 samples of 40 IE per pancreas.

From the tests (FIGS. 1 to 6), it was determined that, when the medium includes a peptone (V1), it provides better results than the serum-free medium V1 and over all of the parameters evaluated, and is comparable to the control medium (VC) containing serum.

Consequently, the medium V1 including a peptone is a formulation optimised for viability (count and apoptosis) and insulin production (secretion of insulin stimulated by glucose and intracellular insulin).

1. Method for preserving a preparation of insulin-secreting cells including:
placing said preparation of insulin-secreting cells in contact with a biological medium including a nutritive product including albumin, and further including a peptone or a mixture of peptones in sufficient quantity to preserve the insulin-secreting cells.

2. Method according to claim 1, wherein the preparation of insulin-secreting cells is a preparation of pancreatic islets.

3. Method according to claim 2, wherein the concentration of pancreatic islets in the biological medium is between 400 and 40,000 islet equivalents/mL.

4. Method according to claim 1, wherein the preparation further includes proteolytic enzymes.

5. Method according to claim 4, wherein the biological medium includes a substrate for said enzymes, and said substrate including said peptone.

6. Method according to claim 1, wherein the biological medium includes between 0.1 and 1% by weight/volume of albumin.

7. Method according to claim 1, wherein the albumin is human serum albumin.

8. Method according to claim 1, wherein the biological medium includes between 0.01 and 10% by weight/volume of peptone.

9. Method according to claim 1, wherein the peptone is a plant peptone.

10. Method according to claim 1, wherein the biological medium further includes polyethylene glycol.

11. Method according to claim 10, wherein the biological medium includes between 0.1 and 3% by weight/volume of said polyethylene glycol.

12. Method according to claim 1, wherein the preparation of insulin-secreting cells is placed in contact with the biological medium for a time period of between 1 and 5 days.

13. Method according to claim 1, further comprising implementing said method during any of isolation, extraction, rinsing, culturing, preservation and transport of the insulin-secreting cells.

14. Composition obtained by the method according to claim 1, including (a) a preparation of insulin-secreting cells and (b) a biological medium with albumin, and one of a peptone and a mixture of peptones.

15. Composition according to claim 14, in which the biological medium includes between 0.1 and 1% by weight/volume of albumin, between 0.01 and 10% by weight/volume of peptone, and between 0.1 and 3% by weight/volume of polyethylene glycol.

16. A Drug for treating pancreatic diseases, including a composition including (a) a preparation of insulin-secreting cells and (b) biological medium with albumin and one of a peptone and a mixture of peptones.
